(19) United States
(12) Patent Application Publication
(10) Pub. No.: US 2018/0214047 A1
(43) Pub. Date: Aug. 2, 2018

(54) Method, Apparatus and Computer-Readable Medium for Assessing Fit in a System for Measuring the Internal Structure of an Object

(71) Applicant: Micrima Limited, Bristol (GB)

(72) Inventor: Mantalena Sarafianou, Bristol (GB)

(21) Appl. No.: 15/749,816

(22) PCT Filed: Jul. 25, 2016
(86) PCT No.: PCT/GB2016/052276

§ 371 Date: Feb. 2, 2018

(30) Foreign Application Priority Data
Aug. 4, 2015 (GB) 1513784.7

(51) Int. Cl. A61B 5/05; A61B 5/00

(52) U.S. Cl. CPC A61B 5/0507; A61B 5/4312; A61B 2562/043; A61B 5/6844; A61B 2562/046; A61B 5/7246

(57) ABSTRACT

The invention provides methods and apparatus for assessing the fit of an object such as a breast to a sensing surface, in a system for investigating the internal structure of an object. The assessment of fit can be carried out in real-time, with the object in situ, enabling the object to be repositioned as necessary before processing is carried out to investigate the internal structure of the object.

### TECHNICAL FIELD

The present invention relates to methods in systems for measuring the internal structure of an object, and particularly relates to methods and apparatus in systems employing microwave radiation to measure the internal structure of an object such as a human breast.

### BACKGROUND

Microwave radar imaging is an imaging modality which exploits the contrasting dielectric properties at tissue boundaries to create a representative 3D energy profile. An object, such as a human breast, is illuminated by a plurality of ultra-wideband signals from a transmit antenna element and reflections, generated at dielectrically contrasting tissue boundaries, are recorded by receiver antenna elements. Tumors can generate significant reflections as they exhibit much higher dielectric properties than adipose tissues due to their significant water content. Synthetic focusing techniques can be used to create an image of any significant dielectric scatterers in the object.

Monostatic imaging systems transmit and receive using the same antenna which can be physically repositioned over the exterior of the object. While monostatic systems can illuminate from a range of angles, the number of acquisition positions is limited as a significant number of measurements must be acquired relatively quickly to minimize displacement of the object from one acquisition to another.

In contrast, multistatic imaging systems employ an array of antennas operating in a Single-In-Multiple-Out manner. That is, each antenna element illuminates the object in turn, while the other antenna elements receive scattered radiation at various angles. The significant number of simultaneous receive elements ensures that significant amounts of data are acquired in a relatively short time frame. However, the number of illuminating paths is limited by the array population and the physical constraints of placing a large number of antennas close to the object.

A problem with any imaging technique that transmits wave energy into an object is that reflections from the surface of the object can cause unwanted signal artifacts. Unfortunately, the location of the surface generally is unknown a priori, is expected to vary from object to object and may vary from antenna to antenna depending on the arrangement of the antenna array and the arrangement of the object with respect to the antenna array.

### SUMMARY

According to a first aspect of the present invention, there is provided a method, in a system for measuring the internal structure of an object, the system comprising an array of transmit/receive antenna elements arranged over a sensing surface. The method comprises: energizing one or more first transmit/receive antenna elements of the array to transmit electromagnetic wave energy towards the object; detecting, at one or more second transmit/receive antenna elements of the array, said electromagnetic wave energy after interaction with the object, thereby generating a plurality of output signals; and generating, based on the plurality of output signals, data which represents the fit of a surface of the object to the sensing surface.

In embodiments of the invention, the method further comprises, responsive to a determination that the object surface is not fitted to the sensing surface, outputting an alert signal requiring that the object be adjusted or rearranged.

In yet further embodiments, the method further comprises: responsive to a determination that the object surface is fitted to the sensing surface, measuring the internal structure of the object based on the plurality of output signals; or, responsive to a determination that the object surface is fitted to the sensing surface, controlling the array of transmit/receive antenna elements to generate a further plurality of output signals, and measuring the internal structure of the object based on the further plurality of output signals.

A number of different algorithms are provided for generating the data representing the fit of the object surface to the sensing surface. For example, one algorithm comprises: based on the plurality of output signals, reconstructing the object surface; and comparing the object surface to the sensing surface. Another algorithm comprises grouping output signals from the plurality of output signals which are expected to be similar; and comparing a particular output signal in a group to other output signals in the group, or to a reference signal generated from the output signals in the group.

In second and third aspects of the invention, a system and a computer-readable medium are provided corresponding to the method recited above.

### BRIEF DESCRIPTION OF THE DRAWINGS

For a better understanding of the present invention, and to show more clearly how it may be carried into effect, reference will now be made, by way of example, to the following drawings, in which:

FIG. 1 is a schematic diagram showing a system according to embodiments of the invention;

FIGS. 2a and 2b are schematic diagrams showing an antenna that may be employed in the system according to embodiments of the invention;

FIG. 3a is a flowchart of a fit assessment process according to an embodiment of the invention;

FIG. 3b is a flowchart of a fit assessment process according to an alternative embodiment of the invention;

FIG. 4 is a flowchart of a first algorithm usable in the fit assessment process shown in FIGS. 3a and 3b; and

FIG. 5 is a flowchart of a second algorithm usable in the fit assessment process shown in FIGS. 3a and 3b.

### DETAILED DESCRIPTION

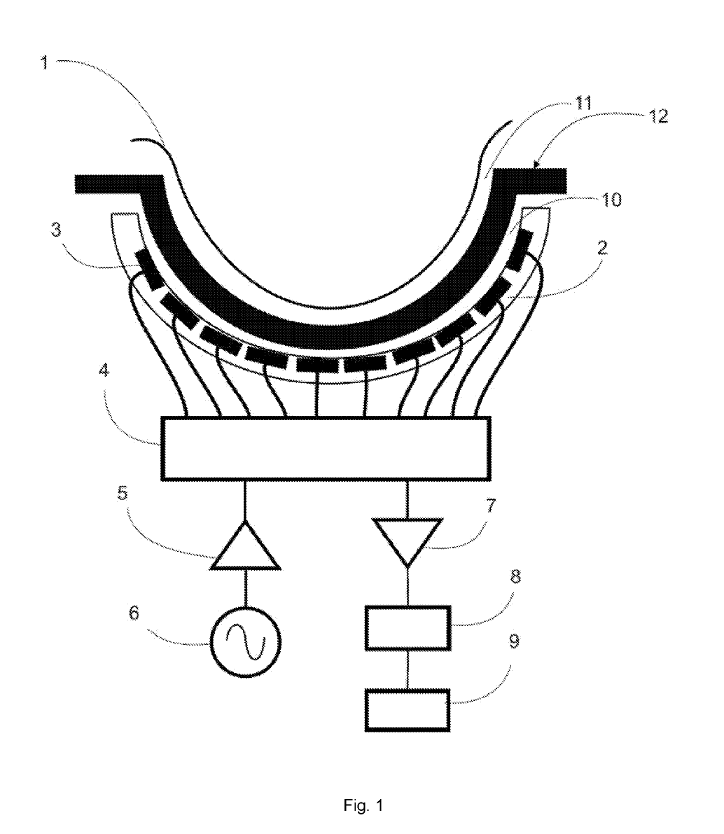

FIG. 1 shows a schematic representation of a system according to embodiments of the invention. The system employs an array of N antennas (e.g. 3), where N>1, arranged over the surface of, or within, a fixed shell 2. For simplicity, FIG. 1 shows the antennas 3 arranged in a linear array; however, it will be appreciated by those skilled in the art that the antennas may generally be arranged in a two-dimensional pattern over the shell 2. The shell 2 may be curved, and in one embodiment at least a portion of the shell corresponds to part of a spherical surface, so as to approximate the shape of a breast 1. In another embodiment, at least a portion of the shell 2 corresponds to a paraboloid. The antennas 3 arranged over this portion of the shell therefore all point to a common focus point, corresponding to the center of the sphere, or the focus of the paraboloid. The shell 2 may have additional portions (and antennas arranged within or on those portions) which do not correspond to part of a spherical or paraboloid surface.

A fixed cup 12 is also provided, and in use a breast 1 (or similar object, such as an imaging phantom) is placed within the cup 12, in order to be imaged or investigated. The cup 12 may have a shape which corresponds substantially to the shape of the shell 2, such that the shell can fit tightly over the cup. For example, the cup may also have at least a portion which corresponds to part of a spherical surface. In one embodiment, the outside of the cup 12 and the inside of the shell 2 may have threaded portions to enable a threaded engagement between the cup and the shell. In use, a layer of dielectric constant controlled fluid may be inserted in the gap 11 between the cup 12 and the breast 1, in order to eliminate or reduce the presence of air in the gap 11 and so improve the coupling of the breast 1 to the microwave radiation generated by the antennas 3. A further layer of dielectric constant controlled fluid may be inserted in the gap 10 between the shell 2 and the cup 12, again so as to improve the coupling between the antennas 3 and the cup 12. The fluid may completely fill the gap 10. The dielectric constant of the fluid may be chosen so as to match a dielectric material used in the antenna 3 and the cup 12, thus minimizing signal loss.

In embodiments of the invention, one or more inserts may be placed inside the cup 12 so as to enable a better fit between the internal surface of the cup and the breast 1. For example, a plurality of such inserts may be provided, each having different shapes and sizes, to enable the system to be better adapted to breasts of different shapes and sizes. The inserts may be made from the same material as the cup 12 (e.g. ceramic).

In one embodiment, the shell 2 is configured to be moveable relative to the cup 12 and therefore the breast 1, e.g. via the threaded engagement between the cup and the shell. For example, the shell 2 might be rotatable about an axis which is substantially perpendicular to the surface on which the antennas 3 are arranged. Where the surface is hemispherical or a paraboloid, the shell 2 may be rotatable about an axis of the hemisphere or the paraboloid. In other embodiments, the shell 2 may alternatively or additionally be translatable over the surface of the cup 12. In still further embodiments, the shell 2 may be fixed (i.e. not movable) with respect to the cup 12.

The array may be operated in multistatic fashion, such that each antenna 3 is arranged to transmit a plurality of signals of microwave radiation in turn, while the received signal at each of the other antennas in the array is recorded. The system thus comprises a switching matrix 4 which selectively couples each antenna 3 to either a tx path or a rx path. The tx path comprises a signal generator 6 coupled to an amplifier 5. When coupled to an antenna via the switching matrix 4, the tx path causes the antenna 3 to transmit a plurality of signals of electromagnetic radiation at a given frequency (for example, in the microwave range). The rx path comprises an amplifier 7 coupled to a detector 8 and a processor 9. The switching matrix 4 arranges for all antennas not coupled to the tx path to be coupled to the rx path (possibly in a time-sharing arrangement), such that the scattered radiation received at each of the non-transmitting antennas is detected and recorded. The system may employ multiple tx and rx paths, such that data can be recorded from multiple antennas simultaneously, and may even comprise a number of tx and rx paths corresponding to the number of antennas, such that data can be recorded from all antennas simultaneously. In alternative embodiments, the array may be operated in monostatic fashion, such that each antenna 3 is arranged to both transmit a plurality of signals of microwave radiation and receive reflected signals resulting from the transmitted signals. In either case, the collected data may then be processed by the processor 9 in a manner set out in more detail below.

The exploitation of the favorable contrast in dielectric properties between normal tissues and malignant tumor depends on radiating and receiving a sufficiently wideband waveform to achieve high resolution. This requires an antenna that radiates well into the breast over a wide band of frequencies. Conventional antennas are obviously not designed to radiate into human tissue, indeed the close proximity of human tissue usually has a detrimental effect on their operation. Additionally the antenna should be inexpensive to construct, suitable for integration into an array and low profile.

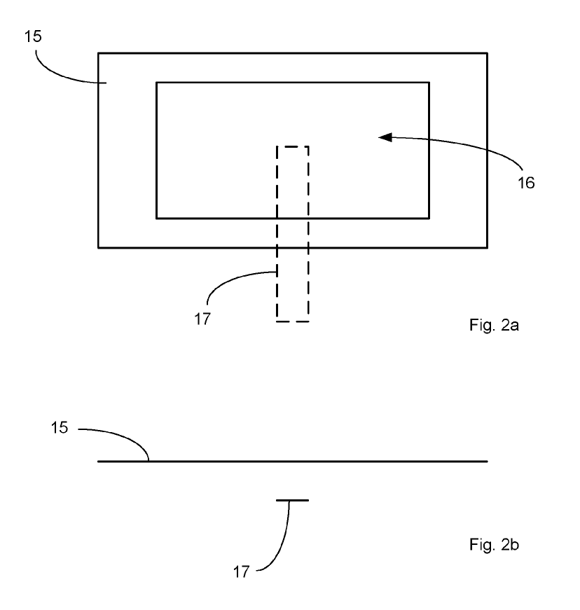

The antenna shown in FIGS. 2a and 2b comprises a slot 16 formed in a conductive element 15, the slot 16 having a rectangular external boundary defined by a substantially closed internal edge of the conductive element 15. The slot 16 is a continuous slot with no internal boundary, the boundary of the slot being completely defined by the internal edge of the conductive element 15.

A microstrip feed line 17 is spaced from the conductive element 15 as can be seen in FIG. 2b, and the distal end of the line 17 is positioned at the geometric center of the slot 16 as can be seen in FIG. 2a. The feed line couples energy to and/or from the slot 16 in a known fashion. The feed line 17 and conductive element 15 are mounted on opposite sides of a dielectric substrate (not shown).

This antenna and other suitable antennas are described in more detail in PCT international publication no WO 2009/060181.

It can be important for systems such as that described above with respect to FIGS. 1, 2a and 2b that the object 1 is correctly fitted to the surface of the cup 12 (or inserts therein). A good fit (i.e. a small, uniform gap between the surface of the object 1 and the cup 12, which is filled with a matching fluid) ensures that signals propagate from the antennas to the object 1 in a strong, consistent manner. In contrast, a poor fit (e.g. due to a mismatch in the shape of the object to the shape of the cup, the presence of air gaps, or the presence of excessive liquid) results in artifacts and anomalies in the final image of the object.

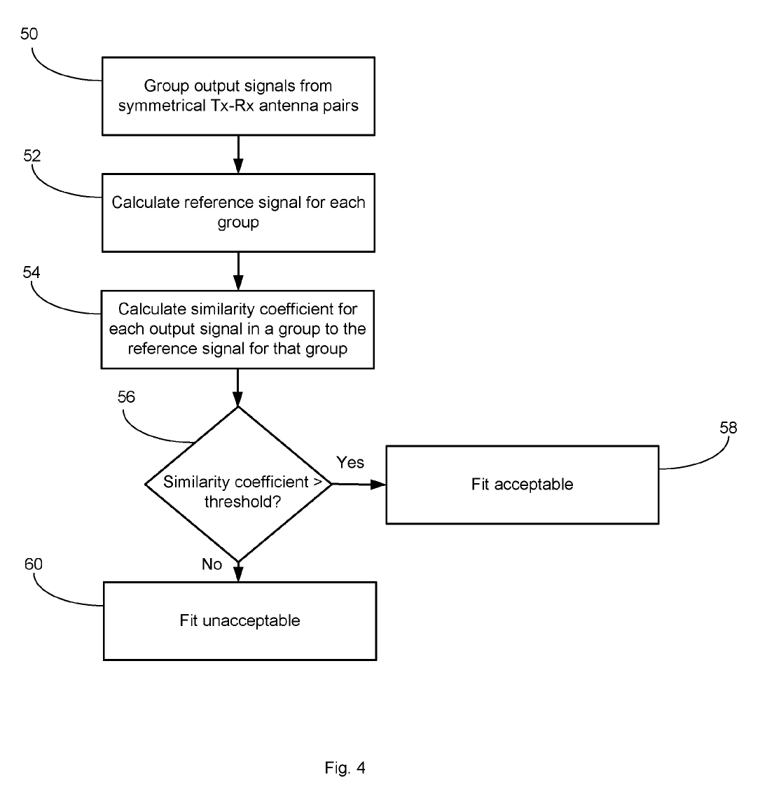

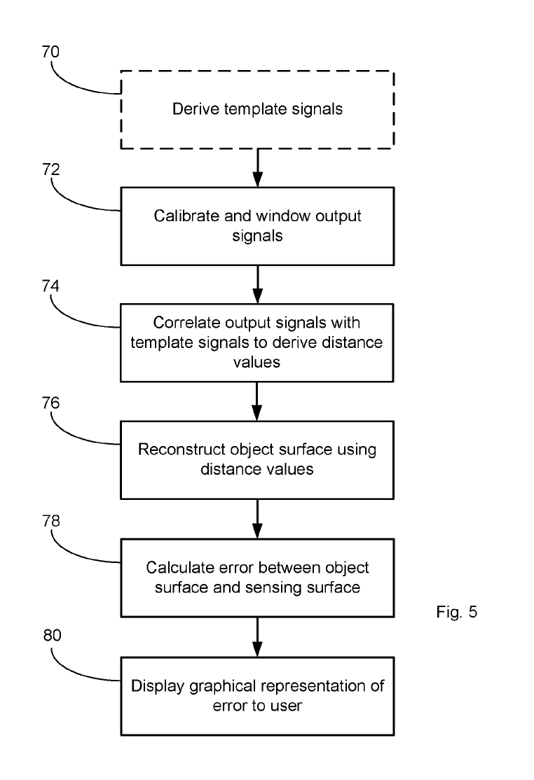

Algorithms set out below measure the fit of the object (particular its surface) to the system (particularly the surface of the cup or inserts) in two ways. One algorithm (set out below with respect to FIG. 5) measures the spatial fit of the object surface to the system, by detecting the surface of the object and comparing it to the known surface of the system. Another algorithm (set out below with respect to FIG. 4) measures the strength of coupling between the antennas 3 and the object 1, and particularly the uniformity of that coupling across the array. This uniformity (or lack of it) is indicative of the spatial fit, in that an object which has a poor spatial fit will generally also have poor uniformity of coupling (in the absence of system faults and the like).

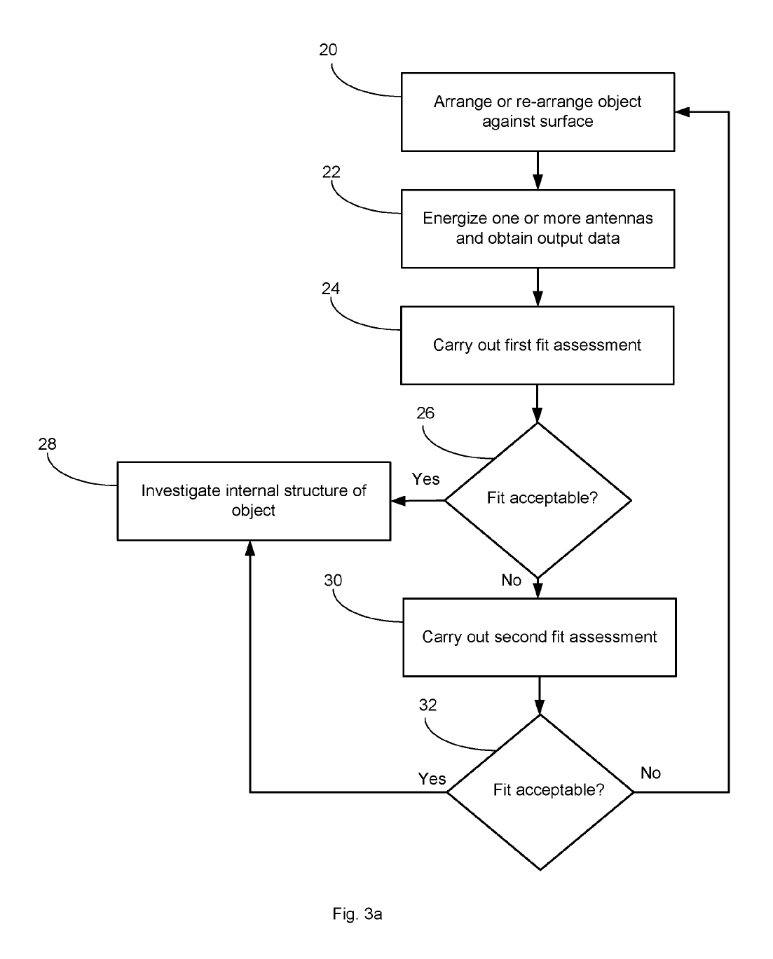

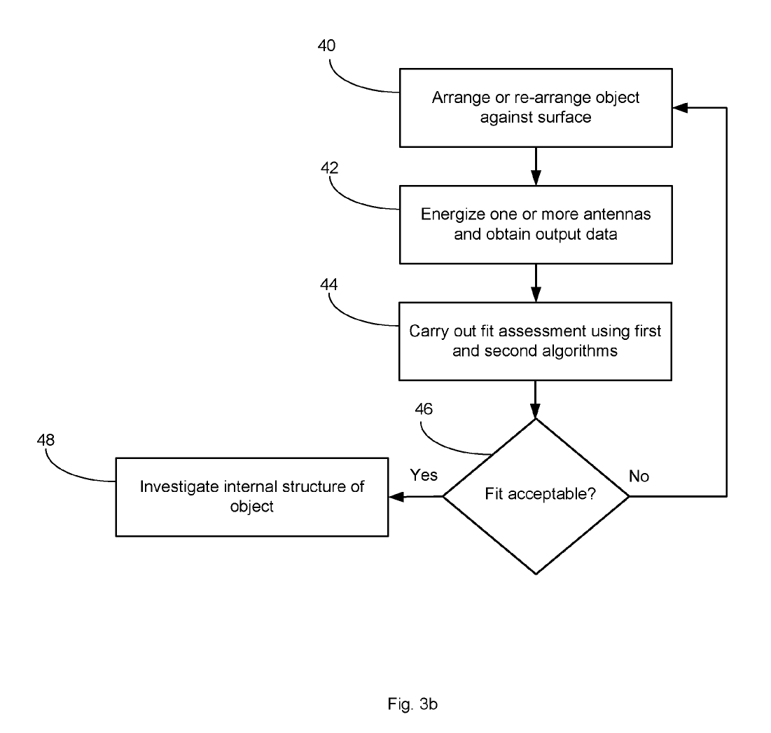

FIG. 3a is a flowchart setting out the steps of a method for assessing the fit of an object, such as a breast, to the system illustrated in FIG. 1.

In step 20, the object is arranged (or re-arranged, see below) against a sensing surface of the system. The object may be a human breast, for example. The sensing surface may be the internal surface of the cup 12, for example, or the surface defined by one or more dielectric constant controlled fluid inserts arranged over the internal surface of the cup 12.

As explained above, it can be important for the correct functioning of the system that the object is correctly fitted to the sensing surface, in that a surface of the object abuts the sensing surface without the presence of air-filled gaps, or unaccounted liquid-filled gaps. Such gaps may cause unwanted artifacts in the signals acquired by the system.

In step 22, one or more antennas 3 of the array 2 are energized to transmit microwave electromagnetic wave energy towards the object, while output signals representative of the wave energy after interaction with the object are received at one or more antennas 3 of the array 2.

In a multistatic mode of operation, a first antenna in the array is energized to transmit electromagnetic wave energy towards the breast. Thus, the antenna is coupled to the tx path in the system, and a plurality of electromagnetic signals are transmitted towards the breast. Substantially simultaneously, the remaining antennas in the array are employed to detect radiation which arises as a result of the transmitted signals. Thus the rx path is connected to each receiving antenna in the array, apart from the transmitting antenna, and the data collected and stored. This process is then repeated such that a second antenna in the array transmits a plurality of signals of electromagnetic wave energy, and so on. The process may be repeated until each antenna 3 in the array 2 has transmitted signals at least once.

In monostatic modes of operation, the transmitting antenna may also be used to collect data; thus, in these embodiments, the rx path may also be connected to the transmitting antenna. As set out above, the rx path may be time-shared amongst the receiving antennas, or there may be multiple rx paths.

The process set out in step 22 thus generates a plurality of output signals, each output signal representative of the radiation detected at an antenna as a result of a plurality of signals of radiation transmitted by an antenna in the array (which may be the same antenna or a different antenna). The radiation detected at each antenna will generally comprise three components: a component arising from mutual coupling between the transmitting and receiving antennas; a component arising from radiation which reflects off the surface of the object 1; and a component arising from radiation which reflects off structures within the object 1 (such as tumors within a breast). The component arising from surface reflections may be used to assess the fit of the object 1 to the sensing surface.

In step 24, a first assessment of the fit of the object to the sensing surface is carried out using the plurality of output signals generated in step 22 and a first fit algorithm. Such processing will typically be carried out by a processor (e.g. processor 9 in FIG. 1), which is programmed with suitable computer-readable code.

In step 26, the output of the first fit algorithm is used to determine if the fit of the object 1 to the sensing surface is acceptable. This may be an automated process, in which the system itself determines that the fit is acceptable, or a user may be involved in assessing the output of the first fit algorithm and determining whether the fit is acceptable on the basis of their experience. If the fit is determined to be acceptable, the process moves to step 28 in which the system can proceed to investigate the internal structure of the object using the output signals acquired in step 22, or new output signals acquired as a result of repeating the process set out above with respect to step 22. For example, the system may generate an image of the internal structure of the object using any of the techniques known in the art, and set out, for example, in PCT international publication WO 2006/085052; Xu Li and S. C. Hagness, A confocal microwave imaging algorithm for breast cancer detection, IEEE Microwave & Wireless Components Lett., vol. 11, pp. 130-2, March 2001; E. C. Fear and M. A. Stuchly, Microwave system for breast tumor detection, IEEE Microwave & Guided Wave Lett., vol. 9, pp 470-2, November 1999; and P. M. Meaney, M. W. Fanning, D. Li, S. P. Poplack and K. D. Paulsen, Clinical prototype for active microwave imaging of the breast, IEEE Trans. on Microwave Theory and Tech., vol. 48, pp. 1841-1853, November 2000.

If the fit is determined not to be acceptable in step 26, the process moves to step 30 in which a second fit assessment of the object to the sensing surface is carried out using the plurality of output signals generated in step 22 and a second, different fit algorithm. Again, the second algorithm will typically be carried out by a processor (e.g. processor 9 in FIG. 1), which is programmed with suitable computer-readable code.

In step 32, the output of the second fit algorithm is used to determine if the fit is acceptable. Again, this process can be automated, or based on a user's assessment of the output. If the fit is determined to be acceptable (in contrast to the determination of the first fit assessment), the process can move to step 28 in which the internal structure of the object is investigated as described above. If the fit is determined not to be acceptable (in agreement with the first fit assessment), an output can be provided to a user indicating that the object should be re-arranged against the sensing surface so as to vary the current fit. The output may be a simple alert message requiring that the object be adjusted or rearranged or, in some embodiments, the output may be qualitative and indicate the region of the object surface at which the fit is unacceptable.

In an embodiment, the first fit algorithm may have relatively lower processing requirements than the second fit algorithm (and so be quicker to carry out), while providing a less accurate assessment of the fit. In this embodiment, the more complex algorithm is only carried out once an indication has been found (via the first algorithm) that the fit is unacceptable in some way. Alternatively, the first fit algorithm may be more complex but with higher accuracy, in which case the second fit algorithm acts as a “sanity check” that the assessment of the first fit algorithm is correct.

FIG. 3b is a flowchart setting out the steps of an alternative method for assessing the fit of an object, such as a breast, to the system illustrated in FIG. 1.

The method begins in step 40, which is identical to step 20 of the method described in FIG. 3a. That is, the object is arranged (or re-arranged, see below) against a sensing surface of the system. Step 42 is again similar to step 22 described with respect to FIG. 3a, where one or more antennas 3 of the array 2 are energized to transmit microwave electromagnetic wave energy towards the object, while output signals representative of the wave energy after interaction with the object are received at one or more antennas 3 of the array 2. The array may be operated in multistatic or monostatic modes.

This generates a plurality of output signals, each output signal representative of the radiation detected at an antenna as a result of a plurality of signals of radiation transmitted by an antenna in the array (which may be the same antenna or a different antenna).

In step 44, an assessment of the fit of the object to the sensing surface is carried out using the plurality of output signals generated in step 42 and first and second fit algorithms. Such processing will typically be carried out by a processor (e.g. processor 9 in FIG. 1), which is programmed with suitable computer-readable code.

In step 46, the outputs of these algorithms are analyzed to see if the first of the object 1 to the sensing surface is acceptable. This may be an automated process, in which the system itself determines that the fit is acceptable, or a user may be involved in assessing the outputs of the first and second algorithms and determining whether the fit is acceptable on the basis of their experience. The fit may be determined to be acceptable if the outputs of both algorithms agree that the fit is acceptable; conversely, the fit may be determined to be unacceptable if the outputs of both algorithms agree that the fit is unacceptable. The designer of the system may determine appropriate rules for handling the situation in which the outputs of the first and second algorithms disagree with each other. For example, one of the algorithms may be more reliable than the other; if the outputs disagree, then the output of the more reliable algorithm may be preferred. Alternatively, the fit may be determined to be unacceptable if at least one of the two algorithms indicates that the fit is unacceptable.

If it is determined that the fit of the object 1 to the sensing surface is acceptable in step 46, the process moves to step 48 in which the system can proceed to investigate the internal structure of the object—as described above with respect to step 28—using the output signals acquired in step 42, or new output signals acquired as a result of repeating the process set out above with respect to step 42. If it is determined that the fit is not acceptable in step 46, the process moves back to step 40, and in an output can be provided to a user indicating that the object should be re-arranged against the sensing surface so as to vary the current fit. The output may be a simple warning message requiring that the object be adjusted or rearranged or, in some embodiments, the output may be qualitative and indicate the region of the object surface at which the fit is unacceptable.

According to embodiments of the invention, therefore, the method involves an assessment of the fit of the object surface to a sensing surface of the system in real time (i.e. with the object in situ). In this way, appropriate adjustments can be made to the fit—if necessary—prior to the complex processing of output signals to investigate the internal structure of the object and, potentially, generation of an image of the object.

The data acquired by the antennas 3 will typically be collected in the frequency domain, and therefore a Fourier transform is first applied to convert each channel of data into the time domain. Note that all data acquired in step 22 is treated in this way. Once the data is transformed, it can be passed to one of the algorithms described below with respect to FIGS. 4 and 5.

FIG. 4 is a flowchart of a first algorithm usable in the fit assessment process shown in FIGS. 3a and 3b. The algorithm provides information on the coupling between the object 1 (particularly its surface) and the surface of the cup 12 (or inserts therein). Such information is indicative of the spatial fit of the surface of the object 1 to the system 1, in that a poor spatial fit (through the presence of air pockets or excessive liquid pockets, or a difference in the shape) will generally result in poor, or non-uniform coupling between the cup and the object 1.

In step 50, output signals generated in step 22 of the previous method are grouped according to whether they are expected to be similar or not. That is, each output signal corresponds to the signal from a particular transmit—receive antenna pair. The array 2 typically has a large number of antenna (e.g. 30, or 60), such that the spatial relationship between one pair of antennas will be similar (i.e. identical or symmetrical) to at least one and potentially several other antenna pairs. Assuming a uniform fit of the object to the sensing surface, the signals arising from these antenna pairs—and particularly those parts of the signals arising from reflections off the object surface—can be expected to be substantially similar. The output signals from these similar antenna pairs are thus grouped together in step 50.

Note that the output signals may be subject to some calibration in order to remove components of the signals resulting from background noise. For example, a set of measurements may be taken while the imaging volume (e.g. the interior of the cup 12) is filled with a fluid medium matching the dielectric permittivity of the cup 12 and the layer of dielectric constant controlled fluid in gap 10. This signal can then be subtracted from the output signal to calibrate and remove components of the signals which do not relate to the surface of the object (as reflections off the surface of the matching medium will be small or non-existent):

y_{cal}(t)=y_{breast}(t)−y₀(t) (1)

where ybreast is the output signal of the receiving antenna, y0 is the signal obtained with a matching medium, and ycal is the calibrated signal. Note that each signal naturally varies over time, and thus the signals must be time-aligned for the calibration to have the intended effect.

A time window may also be applied to the output signals such that only that portion of the signals which relates to surface reflection is subject to processing. For example, the window position and length may be calculated based on the signal path from the transmitting antenna, to the point of reflection (assuming perfect fit) and back to the receiving antenna considering the permittivity of the matching medium and/or the ceramic cup. By looking at only this portion of the output signals, processing can be greatly simplified.

In step 52, a reference signal is calibrated for each group of output signals. For example, in one embodiment, the mean average of the output signals (at each time point t) is used as the reference signal for each group.

In step 54, each output signal within a group is compared with the reference signal for that group. For example, a similarity coefficient may be obtained according to the following equation for the ith signal:

$${r}_{i} = \frac{cov ( F , {\hat{y}}_{i} )}{{σ}_{F} {σ}_{{y}_{i}}} \qquad ( 2 )$$

where the numerator refers to the covariance matrix between the reference signal F(t) and each windowed signal ŷi(t), and σF and σyi are the standard deviations for the signals F(t) and ŷi(t) respectively. Using this formulation, a correlation coefficient value approximating 0 corresponds to a bad fitting while a value approaching 1 corresponds to a good fitting. Alternative methods of comparison will be apparent to those skilled in the art.

The similarity coefficients may be output directly to the user to determine, based on his experience, whether the fit of the object surface to the sensing surface is acceptable or not, in that the coupling between the surface of the object and the ceramic cup 12 (or inserts) is unacceptable. If the system is to determine whether the fit is acceptable, however, the similarity coefficient for each output signal may be compared to a threshold in step 56. For example, the threshold may be set at 0.5, or a value closer to 1. If the similarity coefficient is greater than the threshold, the fit for the antenna pair corresponding to that particular output signal can be deemed acceptable in step 58. That is, the output signal is similar to the other output signals in its group, and this is as expected.

If the similarity coefficient is lower than the threshold, the fit for the antenna pair corresponding to that particular output signal can be deemed unacceptable in step 60. That is, the output signal is not sufficiently similar to other output signals in its group, and this is unexpected as the geometry of the antenna pairs in the group dictates that their corresponding output signals should all be similar. This can be an indication that an air-filled or liquid-filled gap is present between the surface of the object and the sensing surface, i.e. that the fit of the object to the sensing surface is unacceptable.

In embodiments of the invention, step 56 can be repeated for each output signal which has been assigned to a group. The algorithm may conclude that the fit of the object as a whole is unacceptable if one similarity coefficient is below the threshold, or if a predetermined number of similarity coefficients are below the threshold, or if a certain percentage of similarity coefficients are below the threshold. The latter two embodiments of course reduce the likelihood that the algorithm will indicate that the fit is unacceptable.

The algorithm set out in FIG. 4 thus provides an indication of whether or not an object is correctly fitted to the sensing surface. The algorithm can be employed in either step 24 (i.e. the first algorithm) or step 30 (i.e. the second algorithm), and step 44.

FIG. 5 is a flowchart of a further algorithm usable in the fit assessment process shown in FIGS. 3a and 3b. The second algorithm is generally more computationally complex than the algorithm set out in FIG. 4, but can provide a qualitative output indicating where precisely the fit of the object is unacceptable. The algorithm may thus be employed in either step 24 or step 30, and step 44.

The first step of the method is in step 70, where a plurality of template signals are derived. Note that this step is carried out prior to the arrangement of the object against the sensing surface and the acquisition of signals (e.g. as shown in steps 20 and 22), and may be carried out, for example, when the system is initially set up. The imaging volume is filled with a liquid, such as water, which has a dielectric permittivity not matching that of the cup 12. As it is fluid, the liquid “perfectly” fits the sensing surface while, because of its differing permittivity, signals generated by the antennas 3 are subject to strong reflections similar to those of objects such as breasts. Accordingly, a set of measurements is taken with the fluid-filled system and these are referred to herein as “template signals”.

The template signals may be subject to calibration in the same manner as described above with respect to the output signals (i.e. signals taken with a matching medium are subtracted from the template signals in a manner analogous to equation (1)), and may also be windowed in a similar manner so as to avoid unnecessary calculations which do not relate to skin reflections.

It may be the case that an individual template signal is distorted or can have unexpectedly low amplitude. In an embodiment, this problem is mitigated by arranging template signals into groups of signals which arise from symmetrical antenna pairs (as described above with respect to step 50), and averaging the signals such that rather than use an individual template signal for a particular antenna pair, the average is used instead.

In step 72, the output signals are calibrated, in a similar manner to that described above, by subtraction of signals obtained while the imaging volume is filled with a matching medium. The output signals may also be windowed so as to focus only on that time window in which reflections from the surface of the object are expected to occur.

In step 74 each calibrated output signal is correlated with its respective template signal as follows:

$${R}_{cross} = {\sum }_{τ} x ( t ) \cdot {y}^{*} ( t - τ ) \qquad ( 3 )$$

where x(t) is the template signal, y*(t) denotes the complex conjugate of the received signal, τ is the relative delay difference between the two signals and t is the time step.

The total round trip delay trt values are computed from the summation of the relative delay values extracted from the correlation and the reference delay values obtained from the template model geometry. A set of distance values drt is calculated assuming a known speed in the propagation medium based on the computed round trip propagation delay trt values, using the following equation:

$${d}_{rt} = \frac{{c}_{0}}{\sqrt{{ɛ}_{r}}} \cdot {t}_{rt} \qquad ( 4 )$$

where c0 is the speed of light in free space and cr is the dielectric constant of the medium over which the signals propagate.

In step 76 the location of the object surface is derived using the set of distance values drt as follows.

Considering the bistatic mode (or multistatic mode), each drt value is equal to the path made by the incident signal being transmitted by the transmit antenna, reflected off the object surface and recorded at a receiving antenna. Every propagation distance value thus yields a 3-D ellipsoid with the transmitting and receiving antenna positions as its foci. Note that, if employed on monostatic data, the propagation distance value thus yields a sphere.

In 3-D, assume two consecutive antenna positions: transmitter (x1,y1,z1) and receiver (x2,y2,z2). Consider a random point on the ellipse (x,y,z). The distance of that point from the two ellipse foci satisfies the following equation:

\sqrt{(x₁−x)²+(y₁−y)²+(z₁−z)²}+\sqrt{(x₂−x)²+(y₂−y)²+(z₂−z)²}=dᵣᵢ (5)

which is the general equation of a 3-D ellipsoid.

The surface points are found from the intersection of all ellipsoids with a line in the direction (θ,φ) from the center (x0,y0,z0) of the antenna array 2, which in this case coincides with the origin of the co-ordinate system. The coordinates (x0,y0,z0) of the origin are found based on the a priori knowledge of all the antenna positions of the array. Points on the line are described by the following parametric equations:

x=x₁+u(x_{P}_{₂}(ϑ,φ)−x₀) (6-a)

y=y₁+u(y_{P}_{₂}(ϑ,φ)−y₀) (6-b)

z=z₁+u(z_{P}_{₂}(ϑ,φ)−z₀) (6-c)

where the parameter u takes values from the set [0,1] and P2(xP2,yP2,zP2) is defined in the spherical co-ordinate system considering the following equations:

x=x₀+r·sin ϑ·cos φ (7-a)

y=y₀+r·sin ϑ·sin φ (7-b)

z=z₀+r·cos ϑ (7-c)

By substituting the coordinate equations (7a-7c) into equations (6a-6c) and then into equation (5), a quadratic equation is obtained which depends on θ, φ and u. For each (θ,φ) value, the parameter u may be varied to find the intersection of the line with each ellipsoid. Each line may intersect with more than one ellipsoid and hence for each (θ,φ), the boundary point of the object is chosen to be the closest such intersection to the origin. From a physical point of view, the line corresponds to the radius of the object as limited by the selected points of the various ellipsoids. This condition is described by the following equation:

$${min}_{x , y , z} ( distance ) = {min}_{x , y , z} \sqrt{{( x - {x}_{0} )}^{2} + {( y - {y}_{0} )}^{2} + {( z - {z}_{0} )}^{2}} \qquad ( 8 )$$

This condition is applied to the output signals from all the antenna pairs in order to reconstruct the complete object surface.

It may be the case that the output signals include spurious components resulting from reflections off structures other than the object surface (e.g. other components of the system, or structures within the object). As a result, the correlated signal can have several equally-high peaks. For this reason, an additional processing step may be included in some embodiments. For every (θ,φ) pair of values, the two closest reconstruction points—obtained from different ellipsoids—to the origin are averaged to improve the object surface reconstruction quality.

In step 78, the error between the object surface and the sensing surface can be calculated using the following equation (assuming a hemispherical sensing surface):

error(θ,φ)=r_{cup}−{square root over ((x(θ,φ)−x₀)²+(y(θ,φ)−y₀)²+(z(θ,φ)−z₀)²)} (9)

where rcup represents the radius of the ceramic insert used, (x0,y0,z0) denotes the known center of the antenna array 2 and (x(θ,φ), y(θ,φ), z(θ,φ)) refer to every (θ,φ) reconstructed point. A similar equation can be derived for different sensing surfaces.

In step 80, the error values are output graphically to a user, such as in a two-dimensional projection of the sensing surface. Such a visual output provides qualitative information on the fit of the object to the sensing surface, allowing the user to determine whether or not the object is acceptably fitted to the sensing surface and, if not, where on the object surface the fit is unacceptable. In alternative embodiments, the system itself may determine whether the fit is acceptable or not by comparing the error values at each location to a threshold. If the error values exceed the threshold at one or more points, the system may determine that the fit is unacceptable.

The invention thus provides methods and apparatus for assessing the fit of an object such as a breast to a sensing surface, in a system for investigating the internal structure of an object. The assessment of fit can be carried out in real-time, with the object in situ, enabling the object to be repositioned as necessary before processing is carried out to investigate the internal structure of the object.

Those skilled in the art will appreciate that various amendments and alterations can be made to the embodiments described above without departing from the scope of the invention as defined in the claims appended hereto.

1. A method, in a system for measuring the internal structure of an object, the system comprising an array of transmit/receive antenna elements arranged over a sensing surface, the method comprising:
energizing one or more first transmit/receive antenna elements of the array to transmit electromagnetic wave energy towards the object;
detecting, at one or more second transmit/receive antenna elements of the array, said electromagnetic wave energy after interaction with the object, thereby generating a plurality of output signals;
generating, based on the plurality of output signals, data which is indicative of whether a shape of a surface of the object conforms to a shape of the sensing surface and thus the fit of the surface of the object to the sensing surface; and
determining, based on the generated data, whether the surface of the object is fitted to the sensing surface.

2. The method according to claim 1, further comprising:
responsive to a determination that the object surface is not fitted to the sensing surface, outputting an alert signal requiring that the object be adjusted or rearranged.

3. The method according to claim 1, further comprising:
responsive to a determination that the object surface is fitted to the sensing surface, measuring the internal structure of the object based on the plurality of output signals; or
responsive to a determination that the object surface is fitted to the sensing surface, controlling the array of transmit/receive antenna elements to generate a further plurality of output signals, and measuring the internal structure of the object based on the further plurality of output signals.

4. The method according to claim 1, wherein the determining step further includes the step of determining that the object surface is not fitted to the sensing surface if the object surface does not conform to the sensing surface.

5. The method according to claim 1, wherein the determining step further includes the step of determining that the object surface is not fitted to the sensing surface if one or more air- or liquid-filled gaps are present between the object surface and the sensing surface.

6. The method according to claim 1, wherein the generating step further includes the step of generating data which is indicative of the fit of the object surface to the sensing surface, and based on the plurality of output signals, reconstructing the object surface; and
comparing the object surface to the sensing surface.

7. The method according to claim 6, wherein:
the step of comparing the object surface to the sensing surface comprises generating, for each of a plurality of spatial positions, an error value indicative of a mismatch between the object surface and the sensing surface at that spatial position.

8. The method according to claim 1, wherein the generating step, which is indicative of the fit of the object surface to the sensing surface, further includes the steps of:
grouping output signals from the plurality of output signals which are expected to be similar; and
comparing a particular output signal in a group to other output signals in the group, or to a reference signal generated from the output signals in the group.

9. The method according to claim 8, wherein output signals are expected to be similar if they are generated from pairs of first and second transmit/receive antenna elements that are symmetrical with each other.

10. The method according to claim 8, wherein the reference signal is a mean average of the output signals in the group.

11. The method according to claim 10, further comprising determining that the object surface is not fitted to the sensing surface if a correlation coefficient between the particular output signal and the reference signal is below a threshold value.

12. The method according to claim 8, wherein the data which is indicative of the fit of the object surface to the sensing surface is additionally indicative of a coupling between transmit/receive antenna elements in the array and the surface of the object.

13. The method according to claim 1, wherein the sensing surface is substantially hemispherical or substantially a paraboloid.

14. The method according to claim 1, wherein the sensing surface is defined, at least in part, by an internal surface of a cup.

15. The method according to claim 14, wherein the array of transmit/receive antenna elements are arranged over an external surface of the cup.

16. The method according to claim 14, wherein the internal surface is further defined by an insert within the cup.

17. The method according to claim 1, wherein the step of energizing one or more first transmit/receive antenna elements of the array further includes energizing multiple first transmit/receive antenna elements, in turn, to transmit electromagnetic wave energy towards the object.

18. A system for measuring the internal structure of an object, the system comprising:
an array of transmit/receive antennae elements arranged over a sensing surface; and
processing electronics, configured to:
energies one or more first transmit/receive antenna elements of the array to transmit electromagnetic wave energy towards the object;
detect, at one or more second transmit/receive antenna elements of the array, said electromagnetic wave energy after interaction with the object, thereby generating a plurality of output signals;
generate, based on the plurality of output signals, data which is indicative of whether a shape of a surface of the object conforms to a shape of the sensing surface and thus the fit of the surface of the object to the sensing surface; and
determine, based on the generated data, whether the surface of the object is fitted to the sensing surface.

19. The system according to claim 18, wherein the processing electronics is further configured to:
responsive to a determination that the object surface is not fitted to the sensing surface, output an alert signal requiring that the object be adjusted or rearranged.

20. The system according to claim 18, wherein the processing electronics is further configured to:
respond to a determination that the object surface is fitted to the sensing surface, measure the internal structure of the object based on the plurality of output signals; or
respond to a determination that the object surface is fitted to the sensing surface, control the array of transmit/receive antenna elements to generate a further plurality of output signals, and measure the internal structure of the object based on the further plurality of output signals.

21. The system according to claim 18, wherein the sensing surface is substantially hemispherical or substantially parabolic.

22. The system according to claim 18, wherein the sensing surface is defined, at least in part, by an internal surface of a cup.

23. The system according to claim 22, wherein the array of transmit/receive antenna elements are arranged over an external surface of the cup.

24. The system according to claim 22, wherein the internal surface is further defined by an insert within the cup.

25. A computer-readable medium, comprising computer instructions stored thereon which, when carried out by a processor, cause the processor to:
receive a plurality of output signals corresponding to electromagnetic microwave energy detected by a plurality of receivers, arranged over a sensing surface, after interaction with an object; and
generate, based on the plurality of output signals, data which is indicative of whether a shape of a surface of the object conforms to a shape of the sensing surface and thus the fit of the surface of the object to the sensing surface.
